(19) United States
(12) Patent Application Publication
(10) Pub. No.: US 2008/0082360 A1
(43) Pub. Date: Apr. 3, 2008

(54) Inventory monitoring and control applications

(75) Inventors: Kenneth S. Bailey, Newport Beach, CA (US); Paul S. Mula, San Jose, CA (US)

Correspondence Address: Law Office of Scott C Harris, PO Box 1389, Rancho Santa Fe, CA, 92067, US

(73) Assignee: CLEAR-VIEW-TECHNOLOGIES, INC, San Jose, CA (US)

(21) Appl. No.: 11/862,347

(22) Filed: Sep. 27, 2007

Related U.S. Application Data
(60) Provisional application No. 60/848,141, filed on Sep. 29, 2006.
(60) Provisional application No. 60/880,570, filed on Jan. 16, 2007.

(51) Int. Cl. G06Q 50/00; G06Q 10/00

(52) U.S. Cl. 705/2; 705/28

(57) ABSTRACT

Inventory control system which automatically monitors weights of items both before and after dispensing. The difference in weight is used to determine an amount of product that has been dispensed. The amount of product that has been dispensed as compared with the amount of money that the employee takes in for dispensing the product. This can be used to determine employee theft. Embodiments can use various techniques to determine who are the dishonest employees.

This application claims priority from application No. 60/848,141 and from application No. 60/880,570 filed Jan. 16, 2007, the disclosures of which are herewith incorporated by reference.

### BACKGROUND

Previous inventory systems have been used to help determine how many items a store should order. Such systems have historically manually determined the number of products in a store's stock. For example, the products may be manually counted. In the past, stores were forced to “close for inventory”, to allow the products to be counted in this way.

Modern technology has facilitated the inventory process. For example, RFID technology enables electronic inventory taking of pallets to obtain part numbers, date of origin, expiration date and the like. Barcodes have also been used for inventory.

### SUMMARY

The present application describes techniques of automated and substantially real-time inventory control that enables determining amounts and numbers of products.

An aspect takes advantage of this real time inventory control to allow monitoring of dispensed items, as an employee theft deterrent mechanism.

### BRIEF DESCRIPTION OF THE DRAWINGS

In the drawings:

FIG. 1 shows an end to end system in an embodiment which uses wired connection;

FIG. 2 shows a wireless embodiment;

FIG. 3 illustrates a flowchart of operation;

FIG. 4 illustrates an exemplary electronic scale;

FIG. 5 shows an embodiment using a weighing pad array; and

FIG. 6 shows a ganged weighing pad embodiment.

### DETAILED DESCRIPTION

An embodiment described herein teaches an automated inventory system which maintains real-time control over inventoried items to enable more direct real-time monitoring and control over these inventoried items. According to embodiments, the real-time inventory can be used to monitor in real time the dispensing of materials, such as prescription drugs and alcohol.

The inventors recognized a significant issue which has occurred in distribution systems, of a type which are used for portion distribution from a bulk source. Examples of this kind of distribution system include alcohol distribution (e.g., from bars) and prescription drugs.

In a cash business like a bar, employees may give away free drinks, for example, for themselves or for others. The employees may also take in cash. Unscrupulous employees may put the cash in their pocket, rather than in the cash register. An analogous problem can occur in other businesses such as pharmaceutical supply, and can also occur even when the patron is using a payment form other than cash.

The supply companies want to know if a drug is selling out, in order to allow reordering the drug. In addition, however, places where the drugs are dispensed become easy prey for employees to steal the drugs and sell the drugs. In fact, it is believed that drug theft by hospital and drug store employees may account for as much as 48% of all missing and unaccounted for drug inventory discrepancies. The inventors also recognized that manual techniques of maintaining inventory may take months before they turn up any discrepancies, and even once the discrepancy is determined, there is no easy way to determine which employee was responsible for those discrepancies.

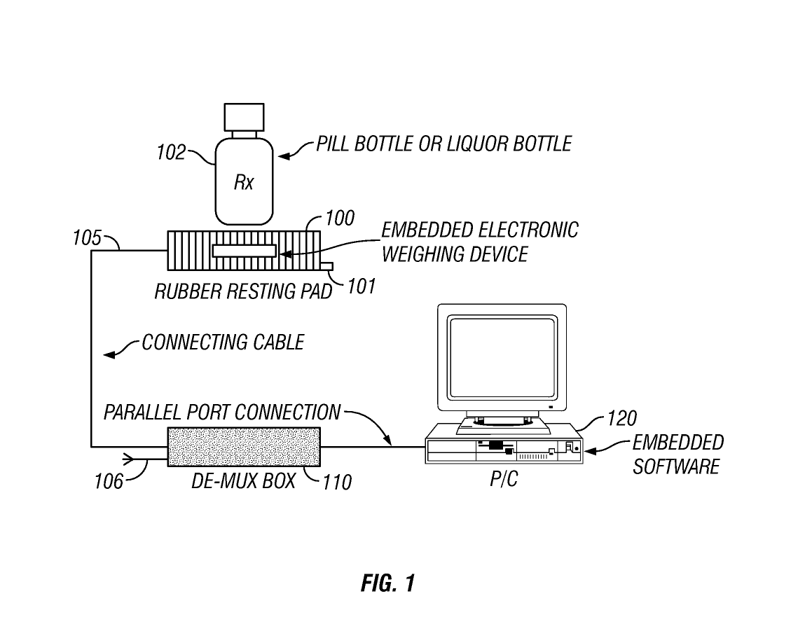

The inventors discovered this problem, and a solution to this problem via computer based real time inventory monitoring. The inventors believe that no one has ever realized this unexpected use and advantage of a real time monitoring system. According to an embodiment, a weight pad 100 is used with an embedded electronic weighing device. The inventoried item is shown here as a prescription bottle 102. The inventoried item could alternatively be for example a liquor bottle, in which case an electronic signal indicative of the weight of the bottle is produced as 105. Multiple different signals are produced, and FIG. 1 shows another signal being received from another pad. These signals are connected to a demux box 110, which converts the signals to a form that allows them to be connected to a port, e.g., parallel port or USB port, of a personal computer 120.

The personal computer monitors the weights of the items on the weight pad 100. For example, dispensed pills which are used may be relatively light. The pad must be sufficiently sensitive to allow detection of weight reduction by the weight of the pills, e.g., 1 mg of pill weight reduction may represent that one pill has been dispensed.

In the embodiment, the personal computer 120 weighs the inventoried item 102 and maintains a running weight. The PC 120 also maintains a list of weights of the different pills; for example if the prescription bottle 102 is Valium, then the PC 120 knows the weight of each Valium pill. Each time a bottle 102 is removed from the scale 100, its weight before and after removal is received by the computer. The computer 120 then produces an indication of the number of pills that were removed, in this way.

A similar technique can be used for sale and dispensing of alcoholic beverages. A manager or owner can determine electronically if the employees are reporting the daily drinks that are served, and at the same time maintaining information indicative of when they need to obtain additional stock.

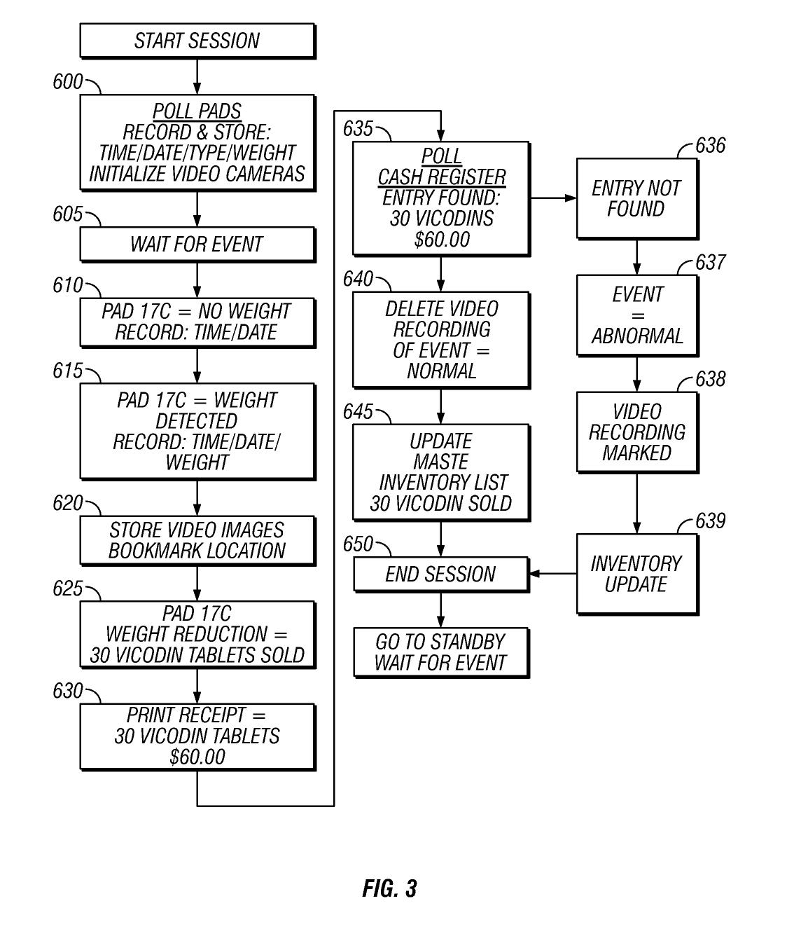

In operation, and as shown and explained with reference to the flowchart of FIG. 3, the personal computer 120 operates to monitor transactions. Each transaction occurs when a bottle 102 is removed from its resting pad. Each pad 100 has a unique address that is associated with its contents such as, for example, that the bottle has Valium 1 mg pills, or alcohol, or the like.

When the bottle 102 is removed from the pad, the weight just before the removal is stored as a “before” weight. The time of removal is also logged. When the bottle is replaced, the newly obtained weight is stored as the “after” weight. The weights measured may be milligrams in case of pills, and weight per ounce of liquor, for example.

The difference between the “before weight” and the “after weight” is divided by the weight of the pills, and the determination is made of how many pills were removed from the bottle. The number of removed pills is stored along with a time of removal, and information indicative of which scale/bottle the pills were removed from.

When the bottle is either empty or almost empty, another monitoring flag is produced, that alerts the owner that it is time to replace the bottle.

The scale can also have a reset button such as 101 which is pressed to signal to the computer that a new bottle has been placed on the pad. In operation, it may be typical for the same size and type of drug or liquor to always be placed on the same pad. However, the pressing of the reset button may be used to signal a full bottle, and may request a re-calibration.

In one embodiment, the weight pads can be connected in a daisy chain series connection, and may include addressable parts, so that the pads can respond to electronic interrogations. Any pad that does not respond to an electronic interrogation may be marked as being in a fault condition.

The weight pad may be formed by any electronic scale item, including, for example, a strain gauge, or a system that uses concentric dielectric materials and tests the strain and condition between those materials. The weight is proportional to the pressure caused on the series of metal cylinders.

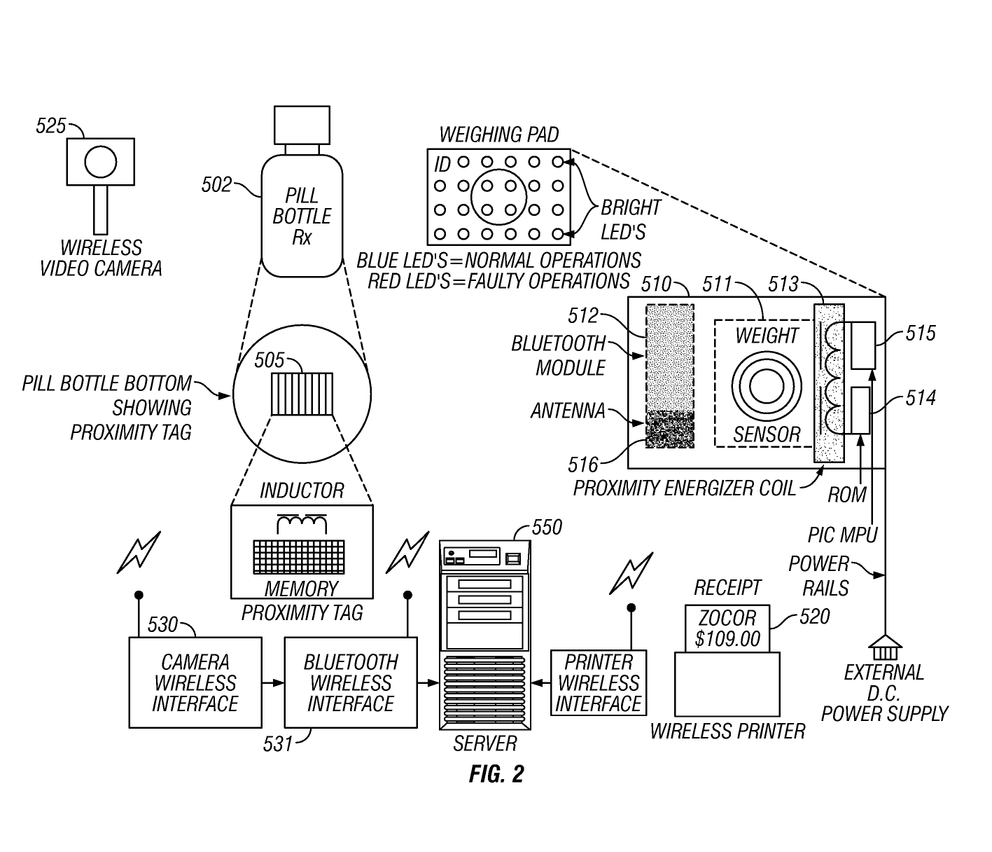

FIG. 2 illustrates an alternative embodiment in which additional controls over the inventory are maintained using a wireless connection. This embodiment uses a wireless connection and further controls and allows further accountability over the actors that are responsible for certain operations.

In the embodiment, the inventoried item 502 includes a proximity tag 505, which can be an RF ID tag or other, associated therewith. The proximity tag indicates certain information about the monitored bottle. The proximity device may store the name and type of the prescription drug, individual weight of pill, the lot number or manufacturer identification code, as well as other information. The proximity tag 505 may include a unique identifier that can be recognized by the computer, such as an inventory number. The inventory number could relate to a database, from which information can be looked up. For example, an inventory number “XZA33” could be stored in a database to represent a bottle of 500 valium, 1 mg each. The tag can alternatively include the information itself such as a unit indication, e.g. “1 mg per pill”.

The proximity tag may be attached to the bottle by a robust adhesive such as epoxy.

In the embodiment, the weighing pad 510 includes structure for reading the proximity information on the tag 505, as well as weighing the bottle. Based on the information in the proximity tag and the weight, a microprocessor unit 515 within the weighing pad 510 may automatically calculate how many pills remain in the bottle 502, or in the alternative embodiment, how much alcohol remains in the bottle. As an alternative, the computer 550 may make this calculation.

As in the first embodiment, the number of pills remaining may be recalculated each time that the bottle is removed from the pad and a master inventory control list is updated to indicate the removed parts.

An embodiment may use a paper receipt detecting system that prints a paper receipt each time there is a reduction in the number of pills, along with a time that this occurred. The printer, shown as 520, may be compared with the cash register receipt at each time of input. Discrepancies in the number of pills, as compared with the received payment, forms an alert.

The alert uses an electronic marker time and date stamp. Based on this alert, information indicative of the person who is handling either or both of the pills or the money is ascertained. In an embodiment, a wireless video camera 525 may continually monitor the proximity of the weighing pad 510. When an alert is caused, the time of the obtained video during which the alert occurred, is either marked or separately transferred to the server. Since the video camera is wireless, the wireless camera interface 530 can send information indicative of the received wireless information to the server computer 550.

This system produces not only determination of when items are being pilfered via inventory control, but also enables determination of the most-likely culprits for having violated the inventory control. For example, whenever more than the desired number of pills have been removed, that is whenever more pills are removed than are actually accounted for, the image obtained wirelessly from the video camera can be used to determine who did the bad act.

Similar techniques can be used for monitoring the dispensing of alcohol. The sale and dispensing of the alcoholic beverages can be coordinated with cash receipts at the end of a period matched to the amount of alcohol that has been served. An absentee bar owner, for example, can thus determine how many free drinks are being given out, and can use the video information to determine who is giving them out, and optionally view that person's behavior during the time of giving them out.

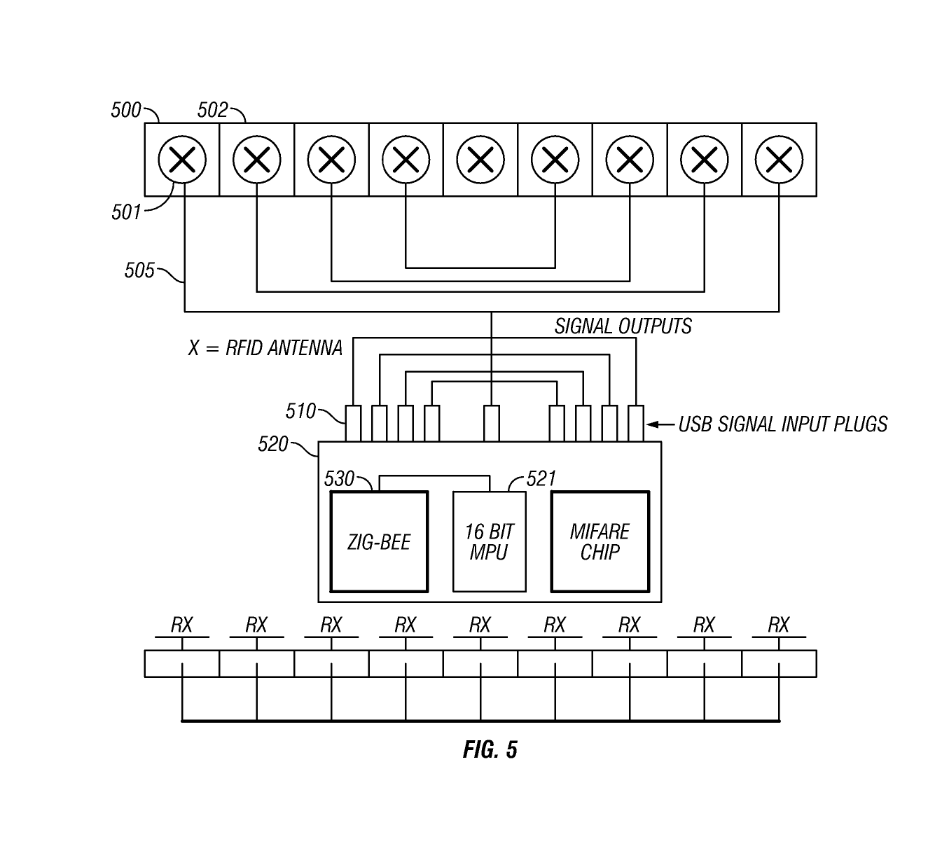

As shown in FIG. 5, the weighing pad 510 may include an electronic weight sensor 511, as well as a wireless module 512 which may transmit via Bluetooth or wireless ethernet, or via any other wireless techniques. A proximity energizer coil 513 may interface with the proximity tag 505, to obtain information therefrom. The tag may be completely passive, or may be energized by the coil 513.

The device may also include its own internal ROM 514 controlling its actions, as well as a microprocessor 515. The module 512, e.g., a Bluetooth module, may have a separate antenna 516. This may transmit to a Bluetooth wireless interface 531, which itself may receive the transmissions from a number of the different weighing pads 510.

As in the above embodiments, the weighing pads 510 are being continuously monitored to determine that they are operating correctly. When correctly operating, the weighing pads light blue LEDs, to indicate normal operations. When not operating properly, the weighing pad displays all red LEDs. The red LEDs may also be used to display an indication that the proximity tag 505 is not being properly read, or that the weight is inconsistent with what it should be, or that there is no item at all on the weighing pad. In this way, someone looking at the system can determine at a glance if the bottles are not properly placed on the pads or if the pads are malfunctioning.

The embodiments may operate according to the flowchart of FIG. 3. The session begins at 600, where all the pads are polled, and the computer 550 records and stores time, date, type, wait of each of the items. The system also initializes the video cameras such as 525. In an embodiment, there may be one video camera placed near each and every item. 605 generically indicates waiting for an event. An event is caused when any item such as 502 is removed from the weighing pad. 610, therefore, illustrates an event, where the item has been removed from the weighing pad, here marked as pad “17C”. The event is detected when the pad sees no weight, or sees a weight less than a specified amount. The time and date when the event occurred is recorded.

At 615, the item is placed back on the pad, the weight is detected, and the time, date and weight is also recorded. 620 illustrates that the video images associated with this event, that is between the beginning time and the ending time, are also noted. These video images may be stored as a bookmark location, or may be separately transferred to the computer as part of the event.

At 625, based on the known weight of the items, the weight reduction is converted into the number of items which were sold. 630 indicates the printing of a receipt at 520, indicating that 30 Vicodin tablets have been sold, and the proper amount that should be taken in at the cash register when 30 Vicodin tablets are sold. In an embodiment, an electronic cash register may be used. The cash register is polled at 635 to determine if 30 Vicodins have been paid for. If the cash register entry matches the weight reduction entry, then the event is set as normal at 640, and the video recording may be deleted.

645 indicates an update to the master inventory list to indicate that 30 Vicodin were sold, and 650 thereafter ends the session.

However, 636 indicates a situation where the entry on the cash register does not match the entry on the weight reduction, meaning that the event is declared as not being normal at 637. At 638, the video recording is marked for later review. An inventory update may also be carried out at 639, to list the actual number of items that have been removed.

The system may print out or otherwise maintain an error indicative of the information. For example, a printer may print an indication of error, date, percentage difference, time, location on the video real, and it dollar amount of discrepancy.

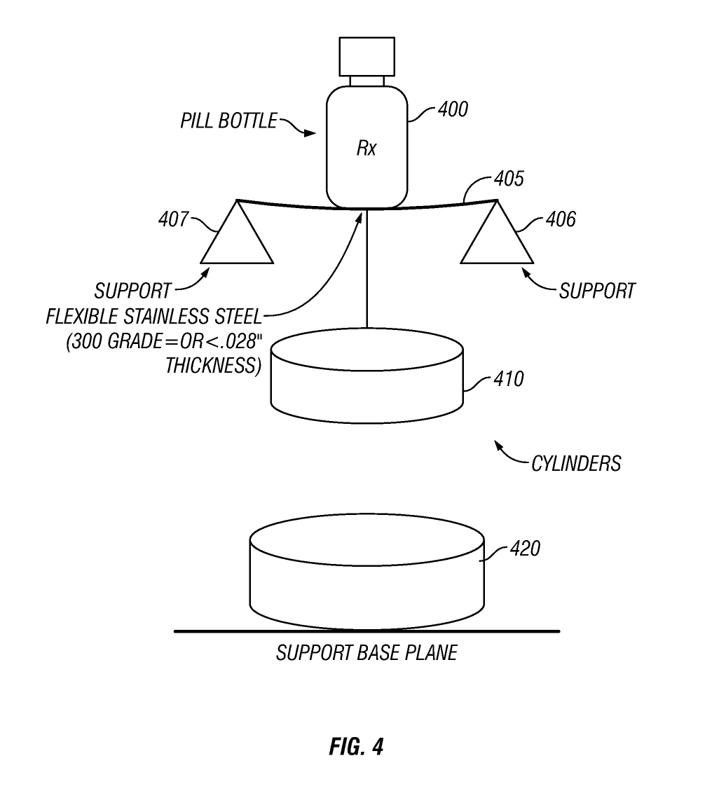

An embodiment shown in FIG. 4 shows a special kind of scale which can be used. This scale shows the pill bottle 400 on a support piece 405 that extends between cantilevers 406, 407. The support peace 405 is flexible stainless steel, for example “300 grade” stainless steel that has less than a 0.028 inch thickness. The support piece 405 forms the top surface of a cylinder 410 that fits within a base plate 420.

FIG. 5 illustrates a weighing pad array. This might be used, for example, in a bar which has multiple different alcoholic beverages, or in a pharmacy that has many different prescriptions to dispense. The array is formed of a number of weighing pads 500, 502, which each can keep a running weight of an item. Each weighing pad such as 500 includes an RF ID part 501. The RF ID part 501 receives the information from the proximity tag on the dispensed item. Signal outputs such as 505 come from the weighing pads, and may be connected via a USB connector 510 to a processing unit 512. The processing unit 520 may include, for example, a microprocessor 521, as well as a wireless network capability 530.

In an embodiment, the wireless network 530 may be via a Zigbee connection or some other wireless system that operates as a point to point repeating system. Alternately, any other wireless protocol described above can be used.

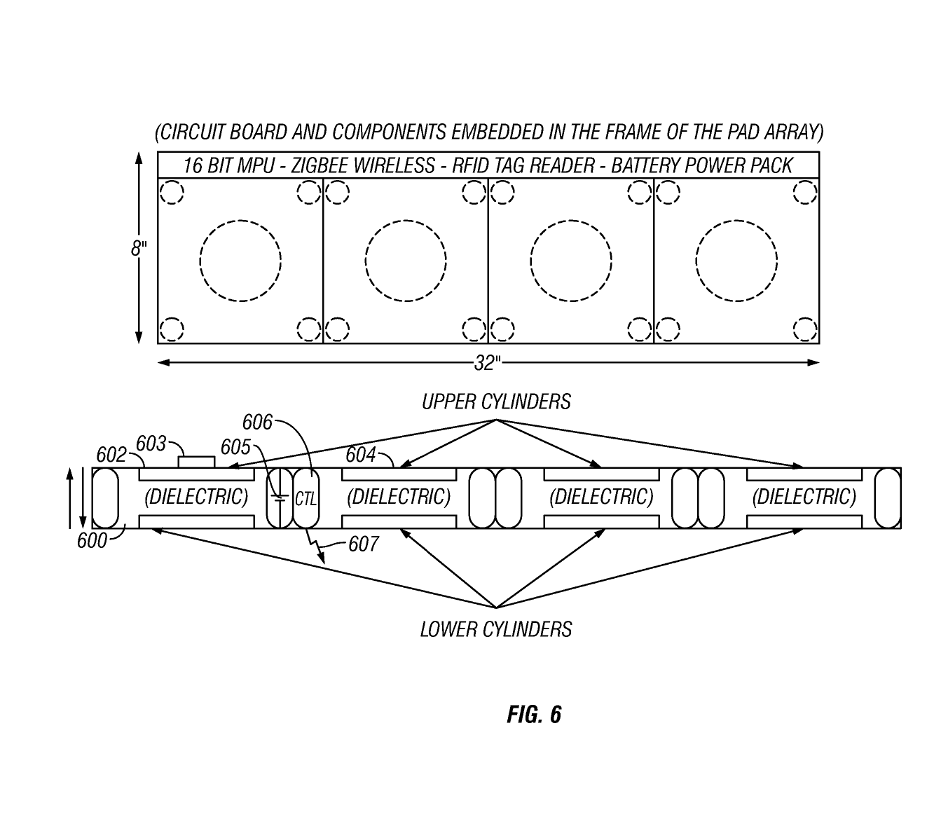

Another embodiment, shown in FIG. 6, forms the weighing pad array of a single continuous element 600, which has a number defining separate weighing stations 602, 604 etc. In this embodiment, each weighing stations also include an RF ID tag reader 603, rechargeable battery 605, as well as a controller 606 which includes a wireless capability 607. When a weight is placed on top of the pads such as 602, the flex in the stainless steel material changes the spacing between the upper and lower cylinders. This in turn changes the capacitance between these plates, and causes an RC tuned circuit to output a signal via the wireless connection 608 indicative of the weight or change of weight of the items.

The embodiment shown in FIG. 6 shows a single continuous pad with separate weighing spots. The device shown in FIG. 6 has four locations to weight four different items. Any item can be placed on the pad, since the readers 603 automatically determines characteristics of the item that has been so placed.

The general structure and techniques, and more specific embodiments which can be used to effect different ways of carrying out the more general goals are described herein.

Although only a few embodiments have been disclosed in detail above, other embodiments are possible and the inventors intend these to be encompassed within this specification. The specification describes specific examples to accomplish a more general goal that may be accomplished in another way. This disclosure is intended to be exemplary, and the claims are intended to cover any modification or alternative which might be predictable to a person having ordinary skill in the art. For example, other data formats, other kinds of scales, etc, may be used.

Also, the inventors intend that only those claims which use the words “means for” are intended to be interpreted under 35 USC 112, sixth paragraph. Moreover, no limitations from the specification are intended to be read into any claims, unless those limitations are expressly included in the claims. The computers described herein may be any kind of computer, either general purpose, or some specific purpose computer such as a workstation. The computer may be an Intel (e.g., Pentium or Core 2 duo) or AMD based computer, running Windows XP or Linux, or may be a Macintosh computer. The computer may also be a handheld computer, such as a PDA, cellphone, or laptop.

The programs may be written in C or Python, or Java, Brew or any other programming language. The programs may be resident on a storage medium, e.g., magnetic or optical, e.g. the computer hard drive, a removable disk or media such as a memory stick or SD media, wired or wireless network based or Bluetooth based Network Attached Storage (NAS), or other removable medium or other removable medium. The programs may also be run over a network, for example, with a server or other machine sending signals to the local machine, which allows the local machine to carry out the operations described herein.

Where a specific numerical value is mentioned herein, it should be considered that the value may be increased or decreased by 20%, while still staying within the teachings of the present application, unless some different range is specifically mentioned. Where a specified logical sense is used, the opposite logical sense is also intended to be encompassed.

What is claimed is:

1. A computerized monitoring system comprising:
an interface that receives information indicative of weights from plural remote elements; and
a processing part, which stores information indicative of a unit quantity of a dispensable item, which determines a weight before dispensing, a weight after dispensing, and produces an output signal indicative of a number of unit quantities which have been dispensed.

2. A system as in claim 1, further comprising an electronic payment system, producing an output indicative of an amount of item that has been dispensed, and which coordinates with said processing part to automatically determine if payment for said number of unit quantities has been received.

3. A system as in claim 2, further comprising a surveillance part, producing an image of a person who does the dispensing, and wherein said processing part automatically produces a time indication associated with said image, when the payment for the number of unit quantities differs from the dispensed number of unit quantities.

4. A system as in claim 1, further comprising a weighing part producing said information indicative of weights, wherein said weighing part is capable of responding to a remote request to determine its condition, and produces a visible indication when an error is detected.

5. A system as in claim 1, further comprising a weighing part producing said information indicative of weights and having a wireless connection between said weighing part and said processing part.

6. A system as in claim 1, wherein said dispensable item is alcohol.

7. A system as in claim 1, wherein said dispensable item is a prescription drug.

8. A system as in claim 1, further comprising a weighing part, and a proximity sensor associated with said weighing part, said proximity sensor reading information from an item located on said weighing part, and automatically determining information indicative of an item on said weighing part.

9. A system as in claim 1, further comprising a weighing part producing said information indicative of weights, and a visible indicator, which visibly indicates whether the weighing part is operating correctly.

10. A system, comprising:
a weighing element, which weighs an item which is located thereon; and
a wireless interface, associated with said weighing element, which automatically wirelessly communicates an output signal indicative of said weight.

11. A system as in claim 10, further comprising a proximity reader, associated with said weighing element, and automatically reading information from a machine-readable part on an item that is associated therewith.

12. A system as in claim 10, wherein said wireless interface comprises a Bluetooth module.

13. A system as in claim 10, wherein said weighing element includes a visible indicator associated therewith, said visible indicator producing a first output indicating that the weighing pad is properly operating, and producing a second output indicating that the weighing element is not properly operating.

14. A system as in claim 10, wherein said weighing element produces a visible indicator associated therewith which produces a first output indicating that an item having a proper weight is on the weighing surface, and produces a second output indicating that an incorrect item having an improper weight is on the weighing surface.

15. A system as in claim 10, further comprising a processor that automatically determines weight information about said item, and automatically determines an amount of said item which has been dispensed.

16. A method, comprising:
weighing at least one dispensable element both before and after dispensing; and
based on said weighing, producing an indication of an amount of said element that has been dispensed; and
automatically comparing said amount with an amount that was indicated as being dispensed by a purchasing database.

17. A method as in claim 16, further comprising determining a time when said amount that was indicated as having been dispensed differs from the amount that has been dispensed, and automatically a producing a time marker indicative of said time.

18. A method as in claim 16, further comprising obtaining a video of an area where said dispensed item was dispensed, and automatically determining a time in said video using said time marker.

19. A method as in claim 16, further comprising automatically determining a characteristic of the dispensable item using a proximity sensor, and wherein said indication is based on said automatically determining.

20. A method as in claim 16 wherein said dispensable item is a prescription drug.

21. A method as in claim 16 wherein said dispensable item is an alcoholic beverage.

22. A method, comprising:
weighing at least one dispensable element at a first time;
detecting that said at least one dispensable element has been removed from a location of weighing;
detecting that said at least one dispensable element has been replaced to said location of weighing, and responsive to detecting that said at least one dispensable element has been replaced to said location of weighing, again weighing said at least one dispensable element; and
based on said weighing and said again weighing, producing an indication of an amount of said element that was removed during a time that said element was removed; and
automatically comparing said amount with an amount indicative of an intended amount of said element that was removed.

23. A method as in claim 22, further comprising automatically detecting characteristics of said dispensable element that are different from a different dispensable element.
